(19) United States
(12) Patent Application Publication
(10) Pub. No.: US 2022/0151807 A1
(43) Pub. Date: May 19, 2022

(54) STENT

(71) Applicant: KAWASUMI LABORATORIES, INC., Oita (JP)

(72) Inventor: Seiichi NAKAYA, Oita (JP)

(21) Appl. No.: 17/431,191

(22) PCT Filed: Feb. 28, 2020
(86) PCT No.: PCT/JP2020/008423

§ 371 Date: Aug. 16, 2021

(30) Foreign Application Priority Data
Mar. 6, 2019 (JP) 2019-040638

(51) Int. Cl. A61F 2/954

(52) U.S. Cl. CPC A61F 2/954; A61F 2002/041

(57) ABSTRACT

A stent is to be placed within a biological lumen (HP) and is provided with: stent parts that have a tubular shape and can expand and shrink in a radial direction that is approximately perpendicular to the axial direction; and transformation means that can transform the stent parts from a reduced diameter state to an expanded diameter state. The transformation means have: first linear members wound around the outer circumferential surfaces of the stent parts; and holding members for holding the first linear members non-detachably from the stent parts. The first linear members are engaged with the holding members to maintain the stent parts in the reduced diameter state, and the engagement is released to transform the stent parts from the reduced diameter state to the expanded diameter state.

### TECHNICAL FIELD

The present invention relates to a stent.

### BACKGROUND ART

There have been known stents that are placed in a stenosis site or an occluded site generated in a living body lumen such as blood vessel, esophagus, bile duct, trachea, and urinary duct, and increase a diameter of a lesion site to maintain an opening state of the living body lumen. In stenting, for example, for a lesion site caused near a hepatic portal, stents should be placed in each of a common hepatic duct, a right hepatic duct, and a left hepatic duct (bile ducts in liver) because the common hepatic duct is branched to the right hepatic duct and the left hepatic duct.

In such a case, conventionally, a plurality of stents e.g. a stent for a main lumen (e.g. common hepatic duct) and stents for branched lumens (e.g. right and left hepatic ducts) are prepared, one stent is inserted into an opening (e.g. mesh of framework part) of the other stent, and these stents are connected to each other while partially overlapping with each other (e.g. see Patent Document 1).

### PRIOR ART DOCUMENT

### Patent Document

Patent Document 1: Japanese Patent Laid-Open No. 2014-138851

### SUMMARY OF THE INVENTION

### Technical Problem

However, in the case of Patent Document 1 or the like, a placement system is required for each stent, and a procedure for placing the stent is complicated, resulting in possibilities of deformation or breakage of the stent or obstruction of a hepatic portal site. On the other hand, when a Y-shaped stent is placed in a branched site of a living body lumen only by one procedure, it is difficult to control timings of release and diameter increasing of each stent part to be placed in a main lumen and branched lumens, and each stent part may not be accurately placed at a placement target site. In addition, not only a Y-shaped stent to be placed on a branched site of a living body lumen but also a general linear stent is demanded to be accurately placed at a placement target site.

An object of the present invention is to provide a stent that can be accurately placed at a placement target site.

### Solution to Problem

The stent according to the present invention is
a stent that is placed in a living body lumen and includes

a stent part having a cylindrical shape and capable of expanding and contracting in a radial direction substantially perpendicular to an axial direction, and

a transformer capable of transforming the stent part from a diameter-decreased state to a diameter-increased state, wherein

the transformer

has a first linear member wound around an outer peripheral face of the stent part, and a holding member for holding the first linear member so as not to drop off from the stent part, and

the stent part is maintained in the diameter-decreased state by engaging the first linear member with the holding member, and the engagement is released to transform the stent part from the diameter-decreased state to the diameter-increased state.

### Advantageous Effect of the Invention

According to the present invention, a stent can be accurately placed at a placement target site.

### BRIEF DESCRIPTION OF THE DRAWINGS

FIG. 1A is a diagram illustrating a configuration of a stent placement system according to an embodiment.

FIG. 1B is a diagram illustrating a configuration of the stent placement system according to the embodiment.

FIG. 2 is a diagram illustrating an appearance of a bile duct stent according to the embodiment.

FIG. 3A is a diagram illustrating an example of a placement manner of the bile duct stent.

FIG. 3B is a diagram illustrating an example of the placement manner of the bile duct stent.

FIG. 4A is a diagram illustrating an example of an engagement manner in a transformer.

FIG. 4B is a diagram illustrating an example of the engagement manner in the transformer.

FIG. 5A is a diagram illustrating a state change during placement of the bile duct stent.

FIG. 5B is a diagram illustrating a state change during placement of the bile duct stent.

FIG. 5C is a diagram illustrating a state change during placement of the bile duct stent.

FIG. 6A is a diagram illustrating a state change during placement of the bile duct stent.

FIG. 6B is a diagram illustrating a state change during placement of the bile duct stent.

FIG. 6C is a diagram illustrating a state change during placement of the bile duct stent.

### DESCRIPTION OF THE EMBODIMENT

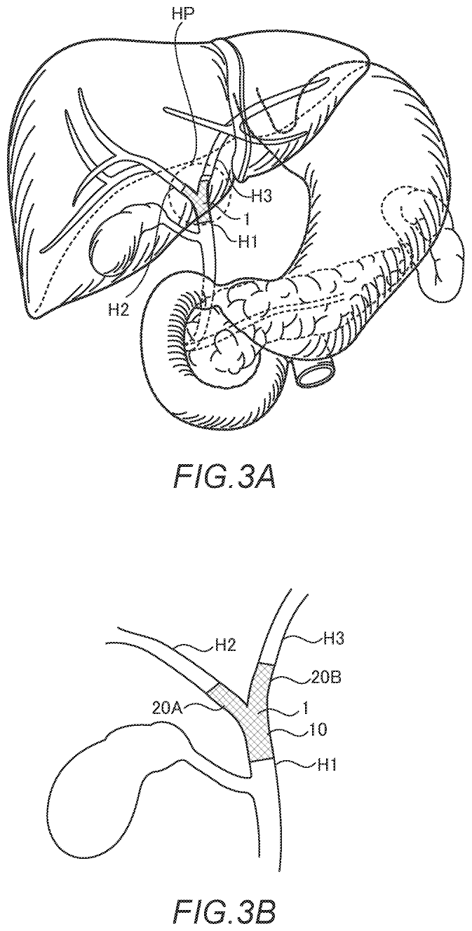

Hereinafter, the embodiment of the present invention will be explained in detail with reference to the figures. In the present embodiment, as an example of the present invention, a bile duct stent 1 will be explained, which is placed in a common hepatic duct H1, a right hepatic duct H2, and a left hepatic duct H3 and used for the purpose of widening a lesion site in the hepatic portal site HP (see FIG. 3A and the like) (e.g. an occlusion site or stenosis site in the hepatic portal site HP) outward in a radial direction for treatment.

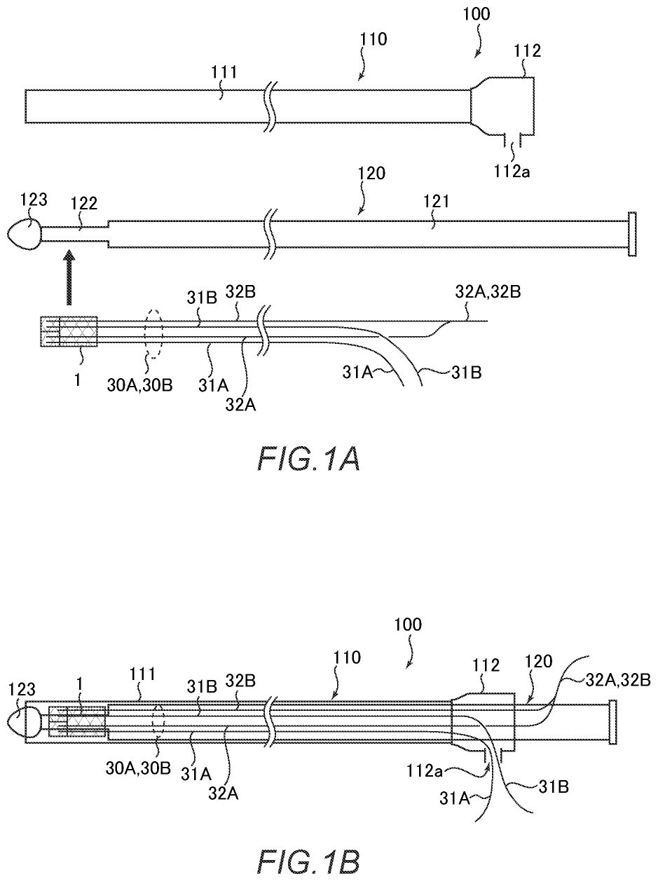

FIG. 1A illustrates a state that a stent placement system 100 is disassembled, and FIG. 1B illustrates a state that the stent placement system 100 is assembled. FIG. 1A and FIG. 1B schematically illustrate a size (length, diameter, etc.), a shape, and the like of each member constituting the stent placement system 100 for the purpose of facilitating understanding of the invention.

When the bile duct stent 1 is placed in the hepatic portal site HP, the stent placement system 100 is used e.g. so as to be inserted into a forceps hole of an endoscope. As illustrated in FIG. 1A and the like, the stent placement system 100 includes a tubular sheath 110, an inner rod 120 disposed inside the sheath 110 and configured to be capable of advancing and retreating along an axial direction of the sheath 110 in the sheath 110, and the bile duct stent 1 accommodated in the sheath 110 in a diameter-decreased state so as to be expandable in the radial direction.

The sheath 110 has e.g. a tubular sheath main part 111 made of a flexible material, and a hub 112 provided on a proximal end side (right side in FIG. 1A and the like) of the sheath main part 111.

The inner rod 120 has e.g. a bar-shaped rod main part 121, a distal tip 123 is provided on the distal end part of the rod main part 121, and a holding part 122 for holding the diameter-decreased bile duct stent 1 is provided slightly closer to the proximal end side than the distal tip 123. Although not illustrated, on the rod main part 121, the holding part 122, and the distal tip 123, e.g. a guide wire lumen through which a guide wire passes, a trigger wire lumen through which a trigger wire for expanding the diameter-decreased bile duct stent 1 on a lesion passes, and the like are formed along an axial direction of the inner rod 120. The rod main part 121, the holding part 122, and the distal tip 123 are made of e.g. various materials having moderate hardness and flexibility such as a resin and a metal, but their detailed explanation is omitted.

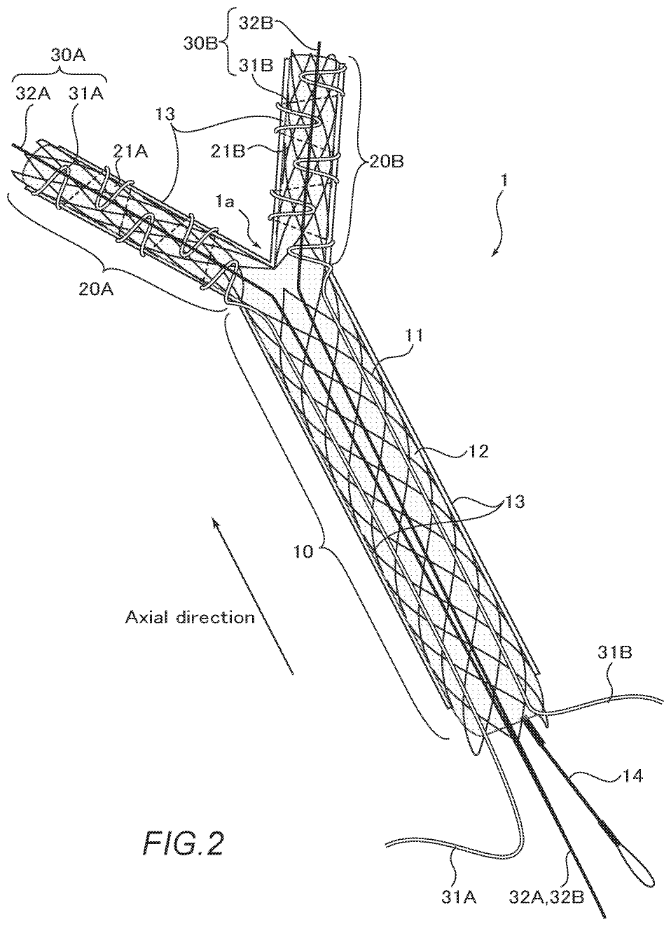

FIG. 2 is a diagram illustrating an appearance of the bile duct stent 1 according to the embodiment. FIG. 3A and FIG. 3B are diagrams illustrating an indwelling state of the bile duct stent 1. FIG. 3B illustrates an enlarged view of the hepatic portal site HP in FIG. 3A.

The bile duct stent 1 is a so-called covered stent. In addition, the bile duct stent 1 has a first stent part 10, and second stent parts 20A and 20B branched from the first stent part 10. As illustrated in FIG. 3A and the like, the first stent part 10 is placed in the common hepatic duct H1, and the second stent parts 20A and 20B are placed in the right hepatic duct H2 and the left hepatic duct H3.

The first stent part 10 and the second stent parts 20A and 20B have a cylindrical shape that demarcates a flow path of bile. In the present embodiment, the second stent parts 20A and 20B have a tube diameter smaller than the first stent part 10 and are connected to each other in series so as to be bifurcated from one end part of the first stent part 10. That means, the bile duct stent 1 has a Y-shape as a whole. An angle of a crotch part la from which the second stent parts 20A and 20B are branched is set depending on a shape of the hepatic portal site HP in which the bile duct stent 1 is placed. In addition, the first stent part 10 may have a straight cylinder shape or a curved shape along a placement site. Furthermore, after the placement, the first stent part 10 may have a curved shape along the common hepatic duct H1.

A first framework part 11 is disposed on the first stent part 10, and second framework parts 21A and 21B are disposed on the second stent parts 20A and 20B respectively. The first framework part 11 and the second framework parts 21A and 21B are stiffening members for maintaining the diameter-increased states of the first stent part 10 and the second stent parts 20A and 20B, and formed e.g. by spirally winding and knitting a metal wire rod. In addition, the first framework part 11 and the second framework parts 21A and 21B have so-called self-expandability, in which a diameter-increased shape is memorized. That means, the first framework part 11 and the second framework parts 21A and 21B are configured to be self-expandable in each radial direction substantially perpendicular to each axial direction, from a diameter-decreased state of contracting inward to a diameter-increased state of expanding outward to demarcate a cylindrical flow path.

The first framework part 11 and the second framework parts 21A and 21B may be connected to or separated from each other at the crotch part 1a. The first framework part 11 and the second framework parts 21A and 21B may have a configuration in which a plurality of frameworks annularly formed while bending a metal wire rod so as to alternately form crest parts and trough parts are arranged at a predetermined interval in each axial direction of 11, 21A and 21B. In addition, the first framework part 11 and the second framework parts 21A and 21B may have a configuration in which one or a plurality of metal wire rods are spirally wound in each axial direction of 11, 21A and 21B while bending the metal wire rods so as to alternately form crest parts and trough parts. In addition, the first framework part 11 and the second framework parts 21A and 21B may be formed by being knitted in a rhombus wire net shape (fence shape) such that a wire rod is folded in a zigzag shape so as to alternately form bending parts (crest parts and trough parts), and the bending parts (crest parts (parts protruding toward one end side in the axial direction) and trough parts (parts protruding toward the other end side in the axial direction)) engage with each other. Furthermore, the first framework part 11 and the second framework parts 21A and 21B may have a laser cut shape obtained by laser-processing a metal cylindrical member.

Examples of a material of the metal wire rod constituting the first framework part 11 and the second framework parts 21A and 21B include known metals or metal alloys typified by a stainless steel, an Ni—Ti alloy (Nitinol), a titanium alloy, and the like. Also, an alloy material having X-ray contrast property may be used. The first framework part 11 and the second framework parts 21A and 21B may be made of a material other than metal materials (e.g. a ceramic, a resin, or the like).

The material, a wire type (e.g. a circular wire rod such as a wire, or an angular wire rod obtained by laser processing), a wire diameter (sectional area), a number and a shape of folds in a circumferential direction (a number and a shape of crest parts), an interval of the wire rod in the axial direction (amount of the framework per a unit length), and the like of the wire rod constituting the first framework part 11 and the second framework parts 21A and 21B are appropriately selected on the basis of flexibility of the first stent part 10 and the second stent parts 20A and 20B, which is required depending on a living body lumen where the stent is placed. Herein, the flexibility refers to the ease of bending the first stent part 10 and the second stent parts 20A and 20B, and is defined particularly by an axial-direction bending rigidity.

In the first stent part 10 and the second stent parts 20A and 20B, a membrane part 12 is arranged along peripheral faces of the first framework part 11 and the second framework parts 21A and 21B. In the present embodiment, the first stent part 10 and the second stent parts 20A and 20B are integrated by integrally forming the membrane part 12.

The membrane part 12 is a film body that forms the flow path for bile. The membrane part 12 may be disposed on the outer peripheral faces and the inner peripheral faces of the first framework part 11 and the second framework parts 21A and 21B so as to sandwich the first framework part 11 and the second framework parts 21A and 21B, or may be disposed on only the outer peripheral faces or only the inner peripheral faces of the first framework part 11 and the second framework parts 21A and 21B.

Examples of a material for forming the membrane part 12 include a fluororesin such as a silicone resin and PTFE (polytetrafluoroethylene), a polyester resin such as polyethylene terephthalate, and the like.

In addition, on the outer peripheral faces of the first framework part 11 and the second framework parts 21A and 21B, extension restricting parts 13 are arranged along each axial direction of the first framework part 11 and the second framework parts 21A and 21B. Specifically, the extension restricting parts 13 are composed of e.g. a rectangular long member, and fixed (e.g. by adhesion, or the like) to the outer peripheral faces of the first framework part 11 and the second framework parts 21A and 21B (e.g. inside of the membrane part 12) so as to extend to both axial-direction end parts of the first framework part 11 and the second framework parts 21A and 21B. In addition, the extension restricting part 13 arranged on the left side of the first stent part 10 and the second stent part 20A in FIG. 2 is continuously and integrally formed, and additionally, the extension restricting part 13 arranged on the right side of the first stent part 10 and the second stent part 20B in FIG. 2 is continuously and integrally formed.

The extension restricting parts 13 are made of e.g. a biocompatible thread (e.g. polyester thread or the like) or a cloth (woven fabric (textile) or knitted fabric), and has a strength capable of restricting extension of the first framework part 11 and the second framework parts 21A and 21B in the axial direction at least within a range that the expandability of the bile duct stent 1 in the radial direction is not impaired.

When the bile duct stent 1 is accommodated in the sheath 110 while decreasing its diameter, the extension restricting parts 13 suppress the extension of the bile duct stent 1 in the axial direction. In addition, when the bile duct stent 1 is released from the sheath 110 to increase the diameters of the first stent part 10 and the second stent parts 20A and 20B, a reduction rate of the bile duct stent 1 in the axial direction is reduced, so that the bile duct stent 1 can be accurately placed at the placement target site on the hepatic portal site HP.

In addition, for example, the extension restricting parts 13 may be provided outside of the membrane part 12. In this case, when the bile duct stent 1 is placed in the hepatic portal site HP, a bile duct wall and the extension restricting parts 13 come into contact with each other, so that the extension restricting parts 13 make inroads into the bile duct wall, and the bile duct stent 1 can be prevented from deviating from the placement position. The extension restricting parts 13 are not necessarily provided.

The other end part (opening end part) of the first stent part 10 is connected with a removal assisting part 14. The removal assisting part 14 is used for removing the bile duct stent 1 placed in the hepatic portal site HP, and has e.g. a loop-shaped engagement part engaged with a hooking implement (snare: recovery member, not illustrated) disposed on a distal end of a recovery catheter. As a wire rod for forming the removal assisting part 14, for example, the same wire rod as for the first framework part 11 can be applied, and the removal assisting part 14 may be formed integrally with the first framework part 11. In addition, a plurality of removal assisting parts 14 may be arranged in the circumferential direction on the opening end part of the first stent part 10.

Additionally, in the bile duct stent 1, transformer 30A and 30B capable of transforming the second stent parts 20A and 20B from the diameter-decreased state to the diameter-increased state are disposed on the outer peripheral faces of the second stent parts 20A and 20B respectively. In the present embodiment, the transformer 30A and 30B are composed of first linear members 31A and 31B wound around the outer peripheral faces of the second stent parts 20A and 20B respectively, and second linear members 32A and 32B engaged with the first linear members 31A and 31B respectively. The transformer 30A and 30B are disposed on the second stent parts 20A and 20B e.g. in a state that the bile duct stent 1 is attached to the inner rod 120, but this configuration is merely an example, and the present invention is not limited to this configuration.

The first linear members 31A and 31B and the second linear members 32A and 32B are made of e.g. a material having a predetermined strength and rigidity, and for example, a suture such as a nylon fiber and a fluorine fiber, a thin metal wire made of a nickel-titanium alloy or a stainless steel, or a string-shaped resin member can be applied. It is preferable that the first linear members 31A and 31B and the second linear members 32A and 32B are made of different materials for improving slidability to facilitate the pulling of the second linear members 32A and 32B. In addition, the first linear members 31A and 32B may be formed in a wide tape shape.

The first linear members 31A and 31B are wound around the outer peripheral faces of the second stent parts 20A and 20B. Specifically, the first linear members 31A and 31B are wound in such a manner that they cannot maintain the wound state by themselves, and held so as not to drop off, by engaging with the second linear members 32A and 32B. That means, in the present embodiment, the second linear members 32A and 32B functionally serves as holding members for holding the first linear members 31A and 31B so as not to drop off from the second stent parts 20A and 20B.

One end sides of the first linear member 31A and 31B are drawn out from e.g. a branch opening 112a provided on the hub 112 (see FIG. 1A and the like). Although not illustrated, for example, one end sides of the second linear member 32A and 32B are drawn out together from an opening provided separately from the branch opening 112a, and can be drawn out while making fine adjustment by turning of one dial, or the like. The second linear members 32A and 32B may be configured such that they can be individually drawn out.

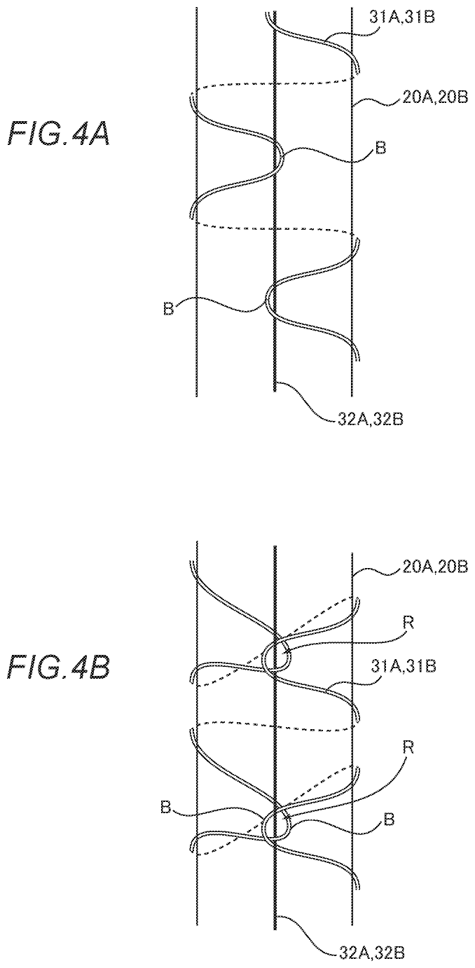

FIG. 4A and FIG. 4B are diagrams illustrating an example of an engagement manner of the transformer 30A and 30B. FIG. 4A and the like illustrate the case where the first linear members 31A and 31B and the second linear members 32A and 32B are disposed on the outer peripheral faces of the diameter-increased second stent parts 20A and 20B for the purpose of facilitating understanding of the engaged manner, but actually, the second stent parts 20A and 20B are tied and their diameters are decreased by winding the first linear members 31A and 31B around the second stent parts 20A and 20B while appropriately applying tension.

As illustrated in FIG. 4A and the like, the first linear members 31A and 31B are wound around the outer peripheral faces of the second stent parts 20A and 20B in the circumferential direction, and bent per one turning and wound in the opposite direction. On the other hand, the second linear members 32A and 32B are arranged along the axial direction of the second stent parts 20A and 20B and engaged with bending parts B formed on the first linear members 31A and 31B. That means, the wound state of the first linear members 31A and 31B is maintained by the engagement with the second linear members 32A and 32B. Thus, once the engagement between the first linear members 31A and 31B and the second linear members 32A and 32B is released, the first linear members 31A and 31B naturally drop off from the second stent parts 20A and 20B.

Specifically, in FIG. 4A, the bending parts B are arranged in the axial direction on the first linear members 31A and 31B. Then, the second linear members 32A and 32B pass through the bending parts B so as to sew the respective bending parts B. For example, the first linear members 31A and 31B are wound while the bending parts B are hooked on the second linear members 32A and 32B arranged along the axial direction of the second stent parts 20A and 20B, so that the first linear members 31A and 31B are engaged with the second linear members 32A and 32B. In this case, the diameters of the second stent parts 20A and 20B are decreased by appropriately pulling the both ends of the first linear members 31A and 31B to apply tension thereto.

Additionally, in FIG. 4B, since the adjacent bending parts B arranged in the axial direction intersect with each other, annular parts R are formed on the first linear members 31A and 31B. Then, the second linear members 32A and 32B pass through the annular parts R. For example, the second linear members 32A and 32B pass through the annular parts R formed by the first linear members 31A and 31B, and the second stent parts 20A and 20B are firmly tied by pulling the both ends of the second linear members 32A and 32B to apply tension thereto, so that the diameters of the second stent parts 20A and 20B are decreased while restraint positions are determined.

In the case of the engagement manner illustrated in FIG. 4A and the like, the engagement between the first linear members 31A and 31B and the second linear members 32A and 32B is easy released by drawing out the second linear members 32A and 32B, and the first linear members 31A and 31B becomes able to drop off from the second stent parts 20A and 20B. As a result, the second stent parts 20A and 20B are released from the diameter-decreased state, and increased in the diameter by the expanding force of the second framework parts 21A and 21B. The wound manner of the first linear members 31A and 31B illustrated in FIG. 4A and the like is merely an example, and another wound manner may be applied.

When attached to the inner rod 120, the bile duct stent 1 is folded in the radial direction while extending in the axial direction so as to be decreased in the diameter, and accommodated in the sheath 110. At this time, the second stent parts 20A and 20B are held in the diameter-decreased state by the transformer 30A and 30B. One end sides of the first linear members 31A and 31B and the second linear members 32A and 32B are drawn out from the opening provided on the sheath 110 (e.g. the branch opening 112a in FIG. 1A).

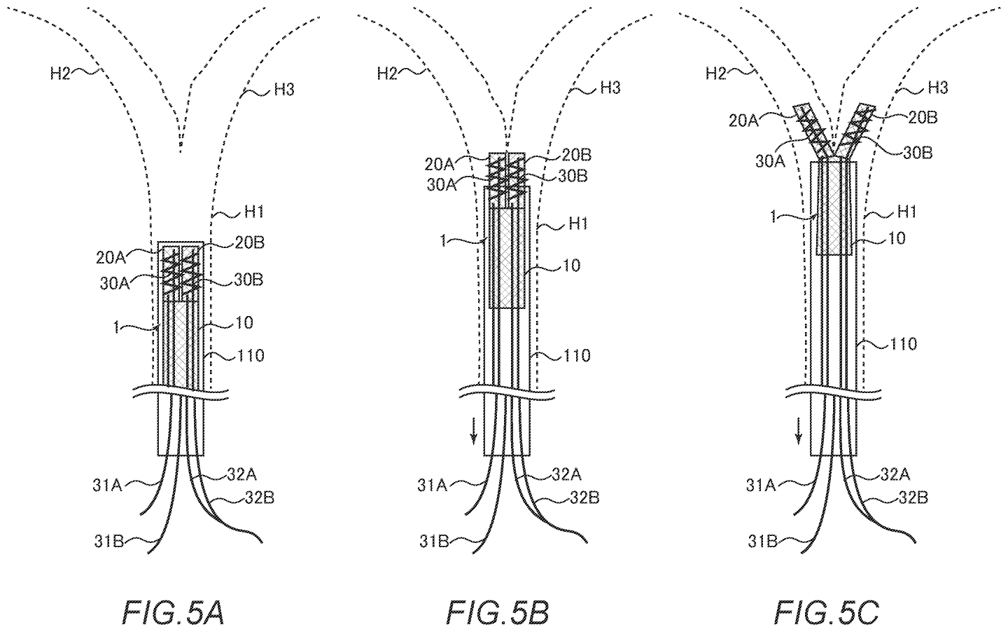

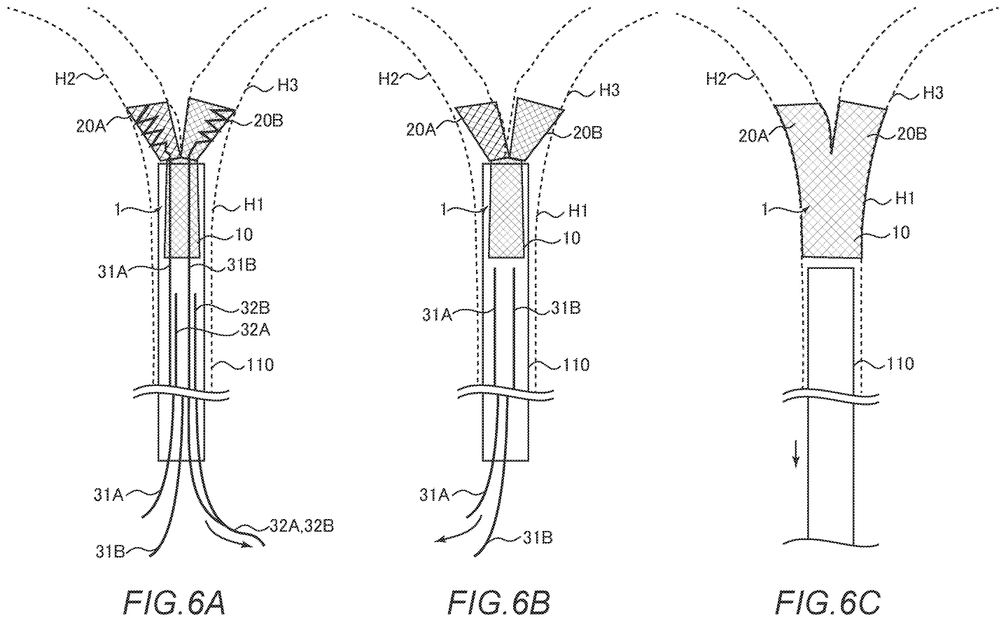

FIG. 5A to FIG. 5C and FIG. 6A to FIG. 6C are diagrams illustrating a state change during placement of the bile duct stent 1. In these figures, the bile duct stent 1 is schematically illustrated, and illustration of the detailed configuration of the first stent part 10 and the second stent parts 20A and 20B is omitted.

When the bile duct stent 1 is placed at the placement target site of the hepatic portal site HP, the sheath 110 and the inner rod 120 are inserted into the hepatic portal site HP from a mouth side along a guide wire (not illustrated) previously introduced into the hepatic portal site HP, and the bile duct stent 1 is positioned such that the distal end of the bile duct stent 1 is in front of the hepatic portal site HP (see FIG. 5A).

Subsequently, the inner rod 120 and the sheath 110 are moved with respect to each other, and positioned on the right hepatic duct H2 and the left hepatic duct H3 respectively while gradually releasing the second stent parts 20A and 20B from the sheath 110 (see FIG. 5B and FIG. 5C). At this time, the first stent part 10 is still accommodated in the sheath 110. In the present embodiment, since the outer peripheral faces of the second stent parts 20A and 20B are restrained by the transformer 30A and 30B, the second stent parts 20A and 20B are maintained in the diameter-decreased state even after being released from the sheath 110.

Subsequently, one end sides of the second linear members 32A and 32B are pulled, and the second linear members 32A and 32B are gradually drawn out (see FIG. 6A). In association with the draw of the second linear members 32A and 32B, the winding of the first linear members 31A and 31B is unwound, and the diameters of the second stent parts 20A and 20B are gradually increased by the expanding force of the second framework parts 21A and 21B. The placement positions of the second stent parts 20A and 20B can be accurately adjusted while gradually drawing out the second linear members 32A and 32B.

Then, in the state that the second stent parts 20A and 20B are placed in the right hepatic duct H2 and the left hepatic duct H3 respectively, the first linear members 31A and 31B are drawn out and recovered (see FIG. 6B), and the sheath 110 is drawn out to release the first stent part 10 of the bile duct stent 1 (see FIG. 6C). The first stent part 10 is increased in the diameter by the expanding force of the first framework part 11, and placed in the common hepatic duct H1. At this time, a reduction rate of the first stent part 10 in the axial direction during the increase in the diameter is reduced by the extension restricting part 13, so that the placement position of the first stent part 10 can be accurately adjusted. In this way, the diameter of the bile duct stent 1 is completely increased from the first stent part 10 to the second stent parts 20A and 20B, and the opening state of the hepatic portal site HP is ensured. Then, although not illustrated, the inner rod 120 is drawn out to place the bile duct stent 1 in the hepatic portal site HP.

As described above, the bile duct stent 1 according to the present embodiment is a stent placed in the hepatic portal site HP (living body lumen), and includes the second stent parts (stent parts) 20A and 20B having the cylindrical shape and capable of expanding and contracting in the radial direction substantially perpendicular to the axial direction, and the transformer 30A and 30B capable of transforming the second stent parts 20A and 20B from the diameter-decreased state to the diameter-increased state. The transformer 30A and 30B have the first linear members 31A and 31B wound around the outer peripheral faces of the second stent parts 20A and 20B, and the holding members (second linear members 32A and 32B) for holding the first linear members 31A and 31B so as not to drop off from the second stent parts 20A and 20B. The second stent parts 20A and 20B are maintained in the diameter-decreased state by engaging the first linear members 31A and 31B with the holding members, and the engagement is released to transform the second stent parts 20A and 20B from the diameter-decreased state to the diameter-increased state. Thereby, the second stent parts 20A and 20B can be maintained in the diameter-decreased state even after being released from the sheath 110, and the second stent parts 20A and 20B can be more accurately positioned with respect to the placement target site of the hepatic portal site HP than the case where the diameters of the second stent parts 20A and 20B are increased after being released. In addition, the diameters of the second stent parts 20A and 20B can be increased only by releasing the engagement of the first linear members 31A and 31B with the second linear members 32A and 32B functionally serving as the holding members. Thus, the bile duct stent 1 can be accurately placed at the placement target site of the hepatic portal site HP.

In addition, the first linear members 31A and 31B are wound around the outer peripheral faces of the second stent parts 20A and 20B in the circumferential direction, and bent per one turning and wound in the opposite direction. As a result, once the engagement with the second linear members 32A and 32B functionally serving as the holding members is released, the first linear members 31A and 31B naturally drop off from the second stent parts 20A and 20B, so that the second stent parts 20A and 20B can be easily transformed to the diameter-increased state by the expanding force of the second stent parts 20A and 20B (second framework parts 21A and 21B).

In addition, the holding members are arranged along the axial direction of the second stent parts 20A and 20B and composed of the second linear members 32A and 32B that pass through the bending parts B formed on the first linear members 31A and 31B. When the second linear members 32A and 32B are drawn out from the bending parts B, the second stent parts 20A and 20B that are tied together with the second linear members 32A and 32B by the first linear members 31A and 31B so as to be decreased in the diameter, are transformed to the diameter-increased state by the transformer 30A and 30B. Specifically, the plurality of bending parts B are arranged along the second linear members 32A and 32B, and the transformer 30A and 30B cause at least either the second linear members 32A and 32B or the second stent parts 20A and 20B to relatively move in the axial direction. Thereby the second linear members 32A and 32B are sequentially drawn out from the plurality of bending parts B, so that the second stent parts 20A and 20B are transformed to the diameter-increased state. Consequently, the second stent parts 20A and 20B can be transformed to the diameter-increased state by a simple operation in which the second linear members 32A and 32B are drawn out.

In addition, the second linear members 32A and 32B pass through the annular part R formed by causing two adjacent bending parts B to intersect with each other. This makes it easier to control the engagement position between the first linear members 31A and 31B and the second linear members 32A and 32B. For example, the second stent parts 20A and 20B are evenly tied in the axial direction to decrease their diameters, so that a desired diameter-decreased state can be formed.

Additionally, in the bile duct stent 1, the diameter-decreased second stent parts 20A and 20B are accommodated in the sheath 110, and the transformer 30A and 30B maintain a part released from the sheath 110 of the second stent parts 20A and 20B in the diameter-decreased state until the engagement between the first linear members 31A and 31B and the holding members (second linear members 32A and 32B) is released. Thereby, a timing of increasing the diameters of the second stent parts 20A and 20B can be controlled by a practitioner, and the second stent parts 20A and 20B can be easily positioned at the placement target site, so that stable procedure is achieved regardless of experience and skill of the practitioner.

The stent parts constituting the bile duct stent 1 include the first stent part 10 placed in the common hepatic duct H1 (first lumen) of the hepatic portal site HP (living body lumen), and the second stent parts 20A and 20B placed in the right hepatic duct H2 and the left hepatic duct H3 (second lumen) branched from the common hepatic duct H1, and the transformer 30A and 30B are provided on at least the second stent parts 20A and 20B. Thereby, the bile duct stent 1 can be easily placed in the hepatic portal site HP as an example of the branched site of the living body lumen only by one procedure.

As described above, the invention made by the present inventors has been specifically explained on the basis of the embodiment, but the present invention is not limited to the embodiment, and can be modified without departing from the gist of the present invention.

For example, in the embodiment, the second linear members 32A and 32B have been illustrated as the holding members for holding the first linear members 31A and 31B, but this configuration is merely an example, the present invention is not limited to this configuration, and may have another configuration. For example, in the state that the first linear members 31A and 31B are wound, the first linear members 31A and 31B may be held so as not to drop off by being fixed to the outer peripheral faces of the second stent parts 20A and 20B with an adhesive.

In addition, although the Y-shaped bile duct stent 1 has been described in the embodiment, the present invention can also be applied to a stent having a branch shape other than the Y-shape, such as a T-shape and a π-shape, and the number of the second stent parts may be 3 or more. Furthermore, the present invention can also be applied to a stent that is not branched but has a straight cylindrical shape.

Additionally, in the embodiment, when the second stent parts 20A and 20B are released from the sheath 110, the first stent part 10 is accommodated in the sheath 110 to maintain the diameter-decreased state of the first stent part 10, but it is also allowed to take a configuration in which the same mechanism as of the transformer 30A and 30B is provided on the first stent part 10, the diameter-decreased state of the first stent part 10 is maintained even after being released from the sheath 110, and then the diameter of the first stent part 10 is increased. In this case, the first stent part 10 is released together with the second stent parts 20A and 20B, and then the bile duct stent 1 can be aligned. The transformer provided on the first stent part 10 can maintain the diameter-decreased state only by tying the first stent part 10 with the linear member without using the holding member, and may be configured to transform the first stent part 10 to the diameter-increased state by drawing out the linear member.

Additionally, in the bile duct stent 1, the first stent part 10 and the second stent parts 20A and 20B may be individually prepared and then connected to each other, or the first framework part 11 and the second framework parts 21A and 21B may be formed of the same wire rod.

Additionally, in the embodiment, for example, the second stent parts 20A and 20B to be placed in the right hepatic duct H2 and the left hepatic duct H3 may be combined with another stent part so as to be extendable.

The present invention can be applied not only to the bile duct stent 1 explained in the embodiment but also to a stent to be placed in a branched site of a living body lumen such as a gastrointestinal lumen and a blood vessel.

The embodiment disclosed in this specification is merely an example in all regards and should be regarded as unrestrictive. The scope of the present invention is stipulated not by the above explanation but by claims, and intended to include meanings equivalent to claims, and all modifications within the scope of claims.

Disclosure contents of the specification, the figures, and the abstracts included in Japanese Patent Application No. 2019-040638 filed on Mar. 6, 2019 are all incorporated in this application.

### DESCRIPTION OF REFERENCE NUMERALS

1 Bile duct stent (stent)
10 First stent part
20A, 20B Second stent part
30A, 30B Transformer
31A, 31B First linear member
32A, 32B Second linear member (holding member)
B Bending part
HP Hepatic portal site (living body lumen)

1. A stent, which is placed in a living body lumen, the stent comprising:
a stent part having a cylindrical shape and capable of expanding and contracting in a radial direction substantially perpendicular to an axial direction, and
a transformer capable of transforming the stent part from a diameter-decreased state to a diameter-increased state, wherein
the transformer has a first linear member wound around an outer peripheral face of the stent part, and a holding member for holding the first linear member so as not to drop off from the stent part, and
the stent part is maintained in the diameter-decreased state by engaging the first linear member with the holding member, and the engagement is released to transform the stent part from the diameter-decreased state to the diameter-increased state.

2. The stent according to claim 1, wherein
the holding member is arranged along the axial direction of the stent part and composed of a second linear member that passes through a bending part formed on the first linear member, and
the transformer transforms the stent part tied together with the second linear member by the first linear member to be brought in the diameter-decreased state, to the diameter-increased state by drawing out of the second linear member from the bending part.

3. The stent according to claim 2, wherein
a plurality of the bending parts are arranged along the second linear member, and
the transformer causes at least one of the second linear member and the stent part to relatively move in the axial direction, and thereby the second linear member is sequentially drawn out from the plurality of bending parts, so that the stent part is transformed to the diameter-increased state.

4. The stent according to claim 1, wherein
the stent part is accommodated in the diameter-decreased state in a sheath, and
the transformer maintains a portion of the stent part released from the sheath in the diameter-decreased state until the engagement between the first linear member and the holding member is released.
